(19) United States
(12) Patent Application Publication
(10) Pub. No.: US 2009/0248122 A1
(43) Pub. Date: Oct. 1, 2009

(54) MULTI-CONDUCTOR RIBBON FOR A LEAD ASSEMBLY OF AN IMPLANTABLE ELECTRIC STIMULATION SYSTEM AND METHODS OF MAKING AND USING

(75) Inventor: Anne Margaret Pianca, Santa Monica, CA (US)

Correspondence Address: Boston Scientific Neuromodulation Corp.;c/o DARBY & DARBY P.C., P.O. BOX 770, Church Street Station, NEW YORK, NY, 10008-0770, US

(73) Assignee: Boston Scientific Neuromodulation Corporation, Valencia, CA (US)

(21) Appl. No.: 12/410,320

(22) Filed: Mar. 24, 2009

Related U.S. Application Data
(60) Provisional application No. 61/040,572, filed on Mar. 28, 2008.

(51) Int. Cl. A61N 1/00; H01R 43/00

(52) U.S. Cl. 607/115; 29/825

(57) ABSTRACT

A lead assembly includes a lead with a plurality of electrodes disposed at a distal end, a plurality of terminals disposed at a proximal end, and an outer lead covering extending along a longitudinal length of the lead from a region proximal to the plurality of electrodes to a region distal to the plurality of terminals. The lead also includes a multi-conductor ribbon disposed within the outer lead covering. The multi-conductor ribbon has a longitudinal length. The multi-conductor ribbon includes a plurality of conductors and a non-conductive insulation. The conductors are aligned longitudinally along the multi-conductor ribbon and the non-conducting insulation encases and insulates each of the conductors, except for the proximal and distal ends of the conductors. Each conductor is electrically coupled to at least one terminal and to at least one electrode.

### CROSS-REFERENCE TO RELATED APPLICATIONS

This application is a utility patent application based on a previously filed U.S. Provisional Patent Application Ser. No. 61/040,572 filed on Mar. 28, 2008, the benefit of which is hereby claimed under 35 U.S.C. § 119(e) and incorporated herein by reference.

### TECHNICAL FIELD

The present invention is directed to the area of implantable electrical stimulation systems and methods of making and using the systems. The present invention is also directed to implantable electrical stimulation systems having a lead that includes a plurality of terminals electrically coupled to a plurality of electrodes by a multi-conductor ribbon, as well as methods of making and using multi-conductor ribbons, leads, and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Deep brain stimulation has also been useful for treating refractory chronic pain syndromes and has been applied to treat movement disorders and epilepsy. Peripheral nerve stimulation has been used to treat chronic pain syndrome and incontinence, with a number of other applications under investigation. Functional electrical stimulation systems have been applied to restore some functionality to paralyzed extremities in spinal cord injury patients. Moreover, electrical stimulation systems can be implanted subcutaneously to stimulate subcutaneous tissue including subcutaneous nerves such as the occipital nerve.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue.

### BRIEF SUMMARY

In one embodiment, a lead assembly includes a lead with a distal end, a proximal end, and a longitudinal length. The lead includes a plurality of electrodes disposed at the distal end, a plurality of terminals disposed at the proximal end, and an outer lead covering extending along the longitudinal length of the lead from a region proximal to the plurality of electrodes to a region distal to the plurality of terminals. The lead also includes a multi-conductor ribbon disposed within the outer lead covering. The multi-conductor ribbon has a first end, a second end, a width, and a longitudinal length. The multi-conductor ribbon includes a plurality of conductors and a non-conductive insulation. The conductors are aligned longitudinally along the multi-conductor ribbon and the non-conducting insulation encases and insulates each of the conductors, except for the proximal and distal ends of the conductors. Each conductor is electrically coupled to at least one terminal and to at least one electrode.

In another embodiment, an electrical stimulating system includes a lead, a control module, and a connector for receiving the lead. The lead has a distal end, a proximal end, and a longitudinal length. The lead includes a plurality of electrodes disposed at the distal end, a plurality of terminals disposed at the proximal end, and an outer lead covering extending along the longitudinal length of the lead from a region proximal to the plurality of electrodes to a region distal to the plurality of terminals. The lead also includes a multi-conductor ribbon disposed within the outer lead covering. The multi-conductor ribbon has a first end, a second end, a width, and a longitudinal length. The multi-conductor ribbon includes a plurality of conductors and a non-conductive insulation. The conductors are aligned longitudinally along the multi-conductor ribbon and the non-conducting insulation encases and insulates each of the conductors, except for the proximal and distal ends of the conductors. Each conductor is electrically coupled to at least one terminal and to at least one electrode. The control module is configured and arranged to electrically couple to electrodes of the lead. The control module includes a housing and an electronic subassembly disposed in the housing. The connector includes a connector housing that defines a first port for receiving the proximal end of the lead. The connector also includes a plurality of connector contacts disposed in the connector housing. The connector contacts are configured and arranged to couple to the terminals disposed at the proximal end of the lead.

In yet another embodiment, a method for forming a lead includes disposing a multi-conductor ribbon into an outer lead covering. The multi-conductor ribbon includes a first end, a second end, and a plurality of conductors extending along the multi-conductor ribbon and separated from one another by insulation. Portions of the insulation are removed to expose each of the conductors at both the first end and at the second end of the multi-conductor ribbon. The exposed first end of each conductor is electrically coupled to at least one terminal. The exposed second end of each conductor is electrically coupled to at least one electrode.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2 is a schematic view of another embodiment of an electrical stimulation system, according to the invention;

FIG. 3A is a schematic view of one embodiment of a proximal portion of a lead and a control module of an electrical stimulation system, according to the invention;

FIG. 3B is a schematic view of one embodiment of a proximal portion of a lead and a lead extension of an electrical stimulation system, according to the invention;

FIG. 4A is a schematic perspective view of one embodiment of a multi-conductor ribbon formed by a plurality of conductors aligned along their longitudinal lengths and coupled together by a shared non-conductive insulation, according to the invention;

FIG. 4B is a schematic transverse cross-sectional view of one embodiment of a multi-conductor ribbon formed by a plurality of conductors aligned along their longitudinal lengths and coupled together by a shared non-conductive insulation, according to the invention;

FIG. 4C is a schematic transverse cross-sectional view of another embodiment of a multi-conductor ribbon formed by a plurality of conductors aligned along their longitudinal lengths and coupled together by a shared non-conductive insulation, according to the invention;

FIG. 5 is a schematic perspective view of one embodiment of the multi-conductor ribbon shown in FIG. 4 with a weakened region extending longitudinally between two conductors of the multi-conductor ribbon, according to the invention;

FIG. 6 is a schematic perspective view of one embodiment of the multi-conductor ribbon shown in FIG. 4 with a weakened region that has been separated to isolate one end of a conductor from the remaining conductors, according to the invention;

FIG. 7A is a schematic perspective view of one embodiment of the multi-conductor ribbon shown in FIG. 4 with portions of insulation ablated to expose each conductor, the ablations configured and arranged to align each conductor with corresponding electrodes when the multi-conductor ribbon is inserted in an outer lead covering, according to the invention;

FIG. 7B is a schematic perspective view of one embodiment of the multi-conductor ribbon shown in FIG. 4 with insulation removed at a second end of the multi-conductor ribbon to expose each the end of each conductor, the exposed conductors aligning with corresponding electrodes when the multi-conductor ribbon is inserted in an outer lead covering, according to the invention;

FIG. 8 is a schematic perspective view of one embodiment of the multi-conductor ribbon shown in FIG. 4 partially wrapped around a mandrel and partially inserted into a lead body, according to the invention;

FIG. 9 is a schematic perspective view of one embodiment of the multi-conductor ribbon shown in FIG. 4 fully wrapped around a mandrel and fully inserted into a lead body, according to the invention;

FIG. 10A is a schematic side view of one embodiment of the multi-conductor ribbon shown in FIG. 4 in a coiled position, according to the invention;

FIG. 10B is a schematic end view of one embodiment of the multi-conductor ribbon shown in FIG. 10A in a coiled position, according to the invention;

FIG. 11A is a schematic end view of one embodiment of a multi-conductor ribbon wrapped once around a mandrel, according to the invention;

FIG. 11B is a schematic end view of one embodiment of a multi-conductor ribbon wrapped multiple times around a mandrel, according to the invention;

FIG. 11C is a schematic end view of one embodiment of a multi-conductor ribbon repeatedly folded into an accordion-like configuration, according to the invention;

FIG. 11D is a schematic end view of one embodiment of a multi-conductor ribbon in a flat configuration, according to the invention;

FIG. 12 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed to implantable electrical stimulation systems having a lead that includes a plurality of terminals electrically coupled to a plurality of electrodes by a multi-conductor ribbon, as well as methods of making and using the leads and electrical stimulation systems.

Suitable implantable electrical stimulation systems include, but are not limited to, an electrode lead (“lead”) with one or more electrodes disposed on a distal end of the lead and one or more terminals disposed on one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; and 6,741,892; and U.S. patent application Ser. Nos. 10/353,101, 10/503,281, 11/238,240; 11/319,291; 11/327,880; 11/375,638; 11/393,991; and 11/396,309, all of which are incorporated by reference.

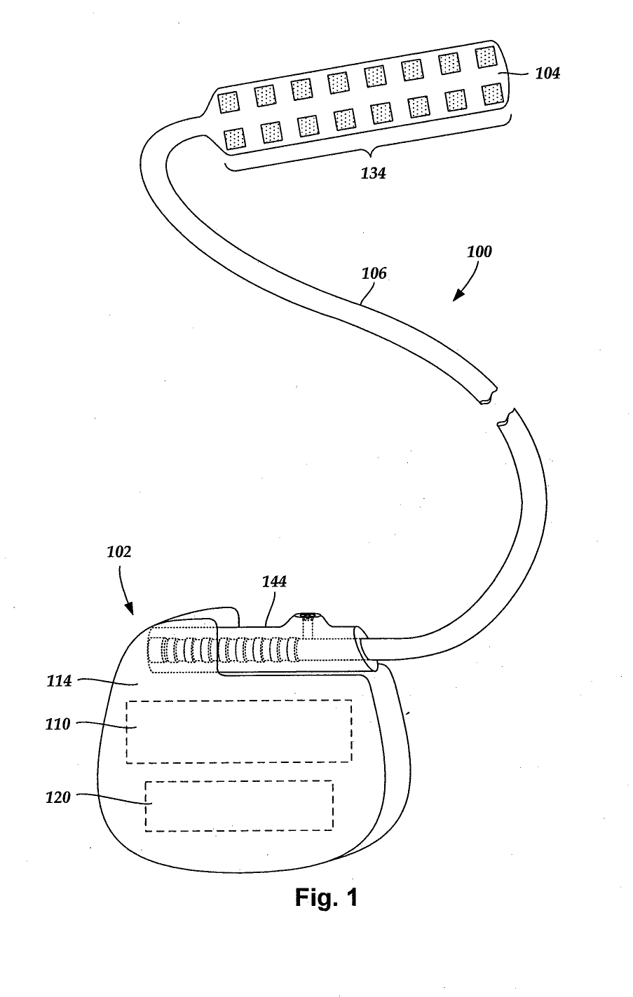

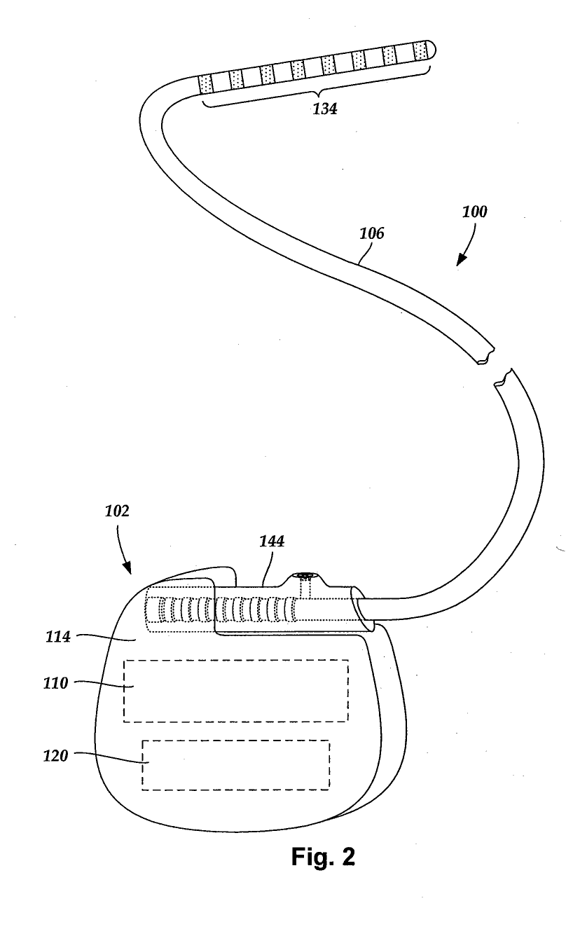

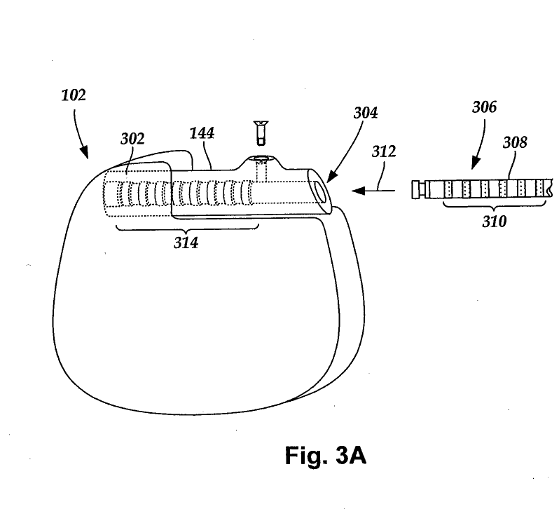

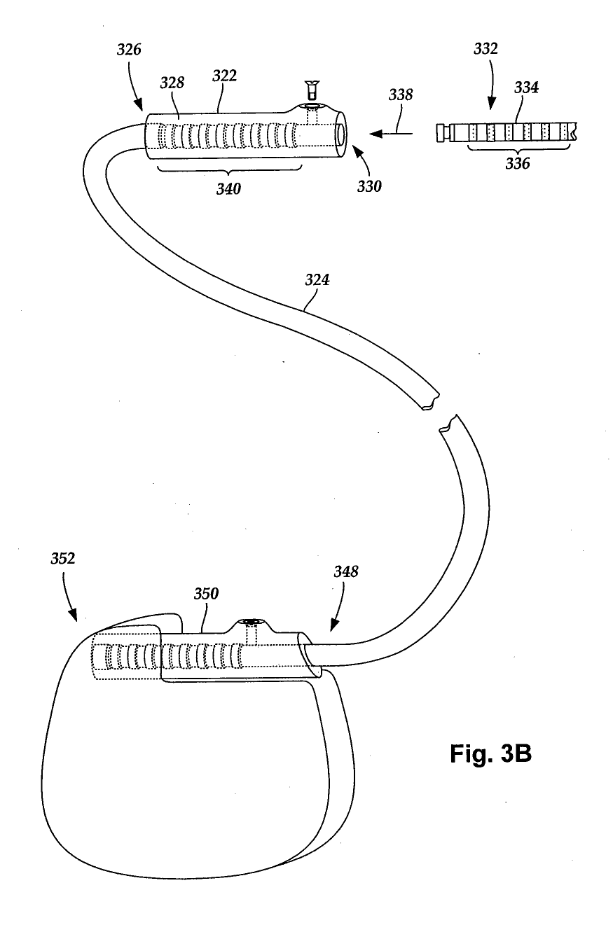

FIG. 1 illustrates schematically one embodiment of an electrical stimulation system 100. The electrical stimulation system includes a control module (e.g., a stimulator or pulse generator) 102, a paddle body 104, and at least one lead body 106 coupling the control module 102 to the paddle body 104. The paddle body 104 and the one or more lead bodies 106 form a lead. The paddle body 104 typically includes an array of electrodes 134. The control module 102 typically includes an electronic subassembly 110 and an optional power source 120 disposed in a sealed housing 114. The control module 102 typically includes a connector 144 (FIGS. 2 and 3A, see also 322 and 350 of FIG. 3B) into which the proximal end of the one or more lead bodies 106 can be plugged to make an electrical connection via conductive contacts on the control module 102 and terminals (e.g., 310 in FIGS. 3A and 336 of FIG. 3B) on each of the one or more lead bodies 106. It will be understood that the electrical stimulation system can include more, fewer, or different components and can have a variety of different configurations including those configurations disclosed in the electrical stimulation system references cited herein. For example, instead of a paddle body 104, the electrodes 134 can be disposed in an array at or near the distal end of the lead body 106 forming a percutaneous lead, as illustrated in FIG. 2. A percutaneous lead may be isodiametric along the length of the lead. In addition, one or more lead extensions 312 (see FIG. 3B) can be disposed between the one or more lead bodies 106 and the control module 102 to extend the distance between the one or more lead bodies 106 and the control module 102 of the embodiments shown in FIGS. 1 and 2.

The electrical stimulation system or components of the electrical stimulation system, including one or more of the lead bodies 106, the paddle body 104, and the control module 102, are typically implanted into the body of a patient. The electrical stimulation system can be used for a variety of applications including, but not limited to, brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof. The number of electrodes 134 in the array of electrodes 134 may vary. For example, there can be two, four, six, eight, ten, twelve, fourteen, sixteen, or more electrodes 134. As will be recognized, other numbers of electrodes 134 may also be used.

The electrodes of the paddle body 104 or one or more lead bodies 106 are typically disposed in, or separated by, a non-conductive, biocompatible material including, for example, silicone, polyurethane, polyetheretherketone (“PEEK”), epoxy, and the like or combinations thereof. The paddle body 104 and one or more lead bodies 106 may be formed in the desired shape by any process including, for example, molding (including injection molding), casting, and the like. Electrodes and connecting wires can be disposed onto or within a paddle body either prior to or subsequent to a molding or casting process. The non-conductive material typically extends from the distal end of the lead to the proximal end of each of the one or more lead bodies 106. The non-conductive, biocompatible material of the paddle body 104 and the one or more lead bodies 106 may be the same or different. The paddle body 104 and the one or more lead bodies 106 may be a unitary structure or can be formed as two separate structures that are permanently or detachably coupled together.

Terminals (e.g., 310 in FIGS. 3A and 336 of FIG. 3B) are typically disposed at the proximal end of the one or more lead bodies 106 for connection to corresponding conductive contacts (e.g., 314 in FIGS. 3A and 340 of FIG. 3B) in connectors (e.g., 144 in FIGS. 1-3A and 322 and 350 of FIG. 3B) disposed on, for example, the control module 102 (or to other devices, such as conductive contacts on a lead extension, an operating room cable, or an adaptor). Conductive wires (not shown) extend from the terminals (e.g., 310 in FIGS. 3A and 336 of FIG. 3B) to the electrodes 134. Typically, one or more electrodes 134 are electrically coupled to a terminal (e.g., 310 in FIGS. 3A and 336 of FIG. 3B). In some embodiments, each terminal (e.g., 310 in FIGS. 3A and 336 of FIG. 3B) is only connected to one electrode 134. The conductive wires may be embedded in the non-conductive material of the lead or can be disposed in one or more lumens (not shown) extending along the lead. In some embodiments, there is an individual lumen for each conductive wire. In other embodiments, two or more conductive wires may extend through a lumen. There may also be one or more lumens (not shown) that open at, or near, the proximal end of the lead, for example, for inserting a stylet rod to facilitate placement of the lead within a body of a patient. Additionally, there may also be one or more lumens (not shown) that open at, or near, the distal end of the lead, for example, for infusion of drugs or medication into the site of implantation of the paddle body 104. In at least one embodiment, the one or more lumens may be flushed continually, or on a regular basis, with saline, epidural fluid, or the like. In at least some embodiments, the one or more lumens can be permanently or removably sealable at the distal end.

In at least some embodiments, leads are coupled to connectors disposed on control modules. In FIG. 3A, a lead 308 is shown configured and arranged for insertion to the control module 102. The connector 144 includes a connector housing 302. The connector housing 302 defines at least one port 304 into which a proximal end 306 of a lead 308 with terminals 310 can be inserted, as shown by directional arrow 312. The connector housing 302 also includes a plurality of conductive contacts 314 for each port 304. When the lead 308 is inserted into the port 304, the conductive contacts 314 can be aligned with the terminals 310 on the lead 308 to electrically couple the control module 102 to the electrodes (134 of FIG. 1) disposed at a distal end of the lead 308. Examples of connectors in control modules are found in, for example, U.S. Pat. No. 7,244,150 and U.S. patent application Ser. No. 11/532,844, which are incorporated by reference.

In FIG. 3B, a connector 322 is disposed on a lead extension 324. The connector 322 is shown disposed at a distal end 326 of the lead extension 324. The connector 322 includes a connector housing 328. The connector housing 328 defines at least one port 330 into which a proximal end 332 of a lead 334 with terminals 336 can be inserted, as shown by directional arrow 338. The connector housing 328 also includes a plurality of conductive contacts 340. When the lead 334 is inserted into the port 330, the conductive contacts 340 disposed in the connector housing 328 can be aligned with the terminals 336 on the lead 334 to electrically couple the lead extension 324 to the electrodes (134 of FIG. 1) disposed at a distal end (not shown) of the lead 334.

In at least some embodiments, the proximal end of a lead extension is similarly configured and arranged as a proximal end of a lead. The lead extension 324 may include a plurality of conductive wires (not shown) that electrically couple the conductive contacts 340 to a proximal end 348 of the lead extension 324 that is opposite to the distal end 326. In at least some embodiments, the conductive wires disposed in the lead extension 324 can be electrically coupled to a plurality of terminals (not shown) disposed on the proximal end 348 of the lead extension 324. In at least some embodiments, the proximal end 348 of the lead extension 324 is configured and arranged for insertion into a connector disposed in another lead extension. In other embodiments, the proximal end 348 of the lead extension 324 is configured and arranged for insertion into a connector disposed in a control module. As an example, in FIG. 3B the proximal end 348 of the lead extension 324 is inserted into a connector 350 disposed in a control module 352.

Conductive wires often include one or more non-conductive materials forming insulation disposed around one or more conductive materials (“conductors”). In some conventional leads, conductors are individually disposed through lumens defined in the lead body. Installation of these conductive wires into a lead body can be slow and tedious. Moreover, once conductive wires are installed in a lead body, identification of individual conductive wires can sometimes be difficult. Identification is increasingly difficult as technological advances allow for a larger number of electrodes to be disposed on a lead which, in turn, will typically increase the number of corresponding conductive wires disposed in the lead.

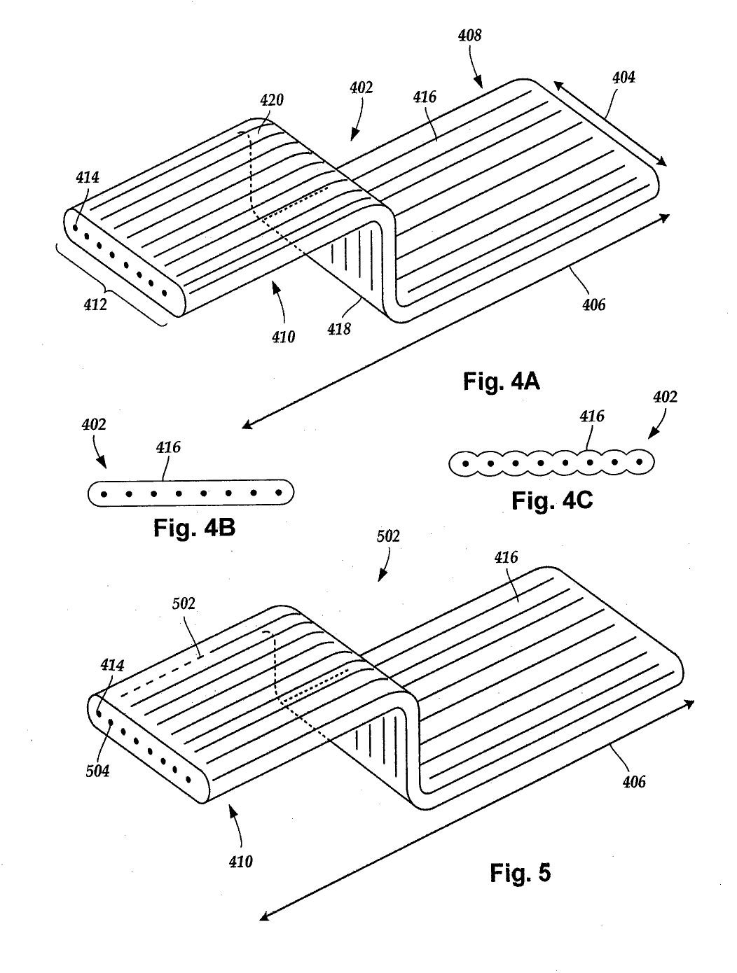

In at least some embodiments, a multi-conductor ribbon can be used instead of individual conductor wires in an electrical stimulation system. The multi-conductive ribbon includes a plurality of conductors coupled together by a shared insulation. FIG. 4 is a schematic perspective view of one embodiment of a multi-conductor ribbon 402. The multi-conductor ribbon 402 has a width, represented in FIG. 4 as a two-headed arrow 404, and a longitudinal length, represented in FIG. 4 as a two-headed arrow 406. Typically, the longitudinal length 406 of the multi-conductor ribbon 402 is much greater than the width 404 of the multi-conductor ribbon 402. The multi-conductor ribbon 402 includes a first end 408 and a second end 410 opposite to the first end 408. A plurality of longitudinally-oriented conductors 412, such as conductor 414, are provided along the longitudinal length 406 and disposed within the shared insulation 416. In at least some embodiments, the conductors 412 are configured and arranged as a single layer of conductors 412. In other embodiments, there can be multiple layers of conductors.

The conductors 412 can be formed using any conductive, biocompatible material. Examples of suitable materials include metals, alloys, conductive polymers, conductive carbon, and the like, as well as combinations thereof. The insulation 416 can be formed using any non-conductive, biocompatible material. Examples of suitable materials include silicone, polyurethane, ethylene, tetrafluoroethylene, polytetrafluoroethylene, polydimethylsiloxane, and the like. The multi-conductor ribbons 402 may be formed in the desired shape by any process including, for example, molding (including injection molding), casting, extrusion, dip coating, and the like.

In FIGS. 4A-10B, eight conductors 412 are shown as an exemplary number of conductors 412 disposed in the multi-conductor ribbon 402. However, any number of conductors 412 can be disposed in a multi-conductor ribbon 402. For example, there can be two, four, six, eight, ten, twelve, fourteen, sixteen, thirty-two, sixty-four, or more conductors 412. As will be recognized, other numbers of conductors 412 may be disposed in a multi-conductor ribbon 402.

In at least some embodiments, the conductors 412 and the insulation 416 are flexible and can be bent in multiple directions. For example, in FIG. 4A, the multi-conductor ribbon 402 includes bends 418 and 420 in the longitudinal axis. As shown in subsequent figures, the multi-conductor ribbon 402 may be bent in other ways as well.

In at least some embodiments, the thickness of the outer coating 416 of the multi-conductor ribbon 402 between the conductors 412 is approximately equal to the thickness of the outer coating in proximity to each of the conductors 412. Accordingly, in some embodiments the transverse cross-sectional shape of the multi-conductor ribbon 402 has approximately-parallel opposing sides, as shown in FIG. 4B. In other embodiments, the outer coating 416 includes depressions, striations, grooves, or the like between adjacent conductors, as shown by the transverse cross-sectional shape of FIG. 4C. The depressions, striations, grooves, or the like may extend along all, or only a portion, of the longitudinal length of the multi-conductor ribbon 402. Although many of the embodiments illustrated in the Figures have the cross-sectional arrangement illustrated in FIG. 4B, it will be understood that such embodiments can be modified to include the cross-sectional arrangement illustrated in FIG. 4C.

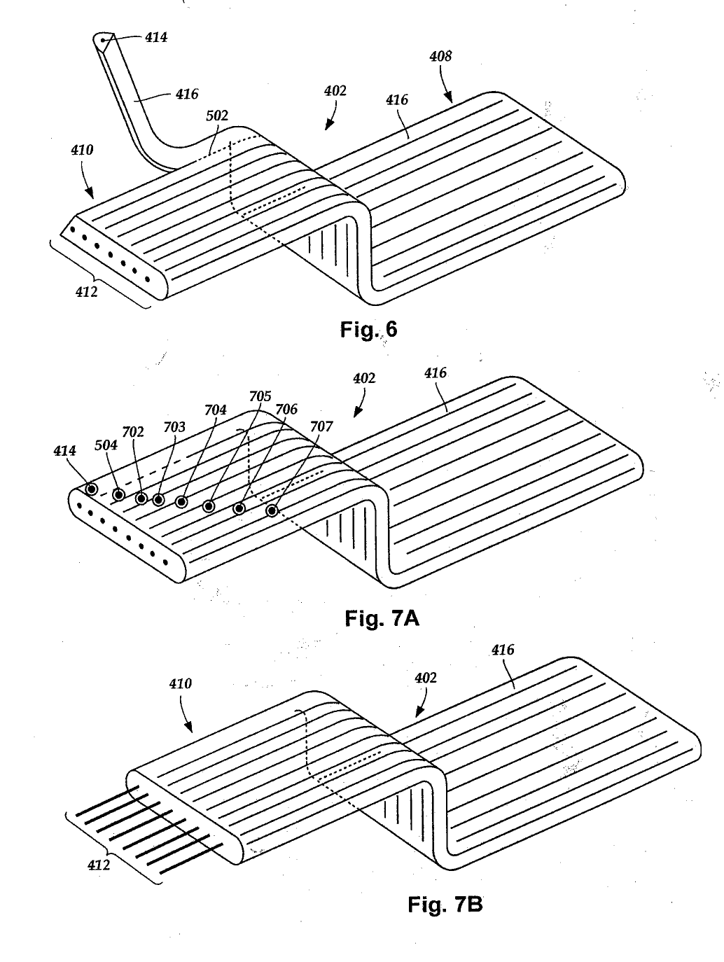

In at least some embodiments, weakened regions may be formed in one or more desired locations in the insulation 416 to facilitate bending or folding or to facilitate separation of one or more portions of the multi-conductor ribbon 402 from other portions of the multi-conductor ribbon 402. For example, in some embodiments, weakened regions may be formed in the insulation 416 that extend along the longitudinal length 406 of the multi-conductor ribbon 402. FIG. 5 is a schematic perspective view of one embodiment of the multi-conductor ribbon 402 with a weakened region 502, shown in FIGS. 5 and 6 as a dashed line, formed in the insulation 416 of the second end 410 and extending along a portion of a longitudinal length 406 between the conductor 414 and an adjacent conductor 504 of the multi-conductor ribbon 402. In some embodiments, a weakened region may be formed in the insulation 416 of the first end 408 in addition to, or instead of, the second end 410. In some embodiments, weakened regions may extend the entire length of the multi-conductor ribbon 402 from the first end 408 to the second end 410. In some embodiments, weakened regions may be disposed in other portions of the multi-conductor ribbon 402 besides the first end 408 or the second end 410 and may extend in directions other than longitudinally along the multi-conductor ribbon 402 and may also extend in a linear or non-linear manner. In some embodiments, a weakened region is formed by one or more perforations.

In some embodiments, a portion of the multi-conductor ribbon can be separated from other portions of the multi-conductor ribbon by separating the insulation along a weakened region. FIG. 6 is a schematic perspective view of one embodiment of the multi-conductor ribbon 402 with a partial separation along the weakened region 502. In FIG. 6, the weakened region 502 is partially separated at the second end 410, thereby separating a portion of the conductor 414 from the remainder of the multi-conductor ribbon 402. The partially-separated conductor 414 includes a portion of the insulation 416 encasing a longitudinal length of the separated portion of the partially-separated conductor 414. In at least some embodiments, conductors may be separated along weakened regions disposed at the first end 408 of the multi-conductor ribbon instead of, or in addition to, being separated at the second end 410.

In FIG. 6, the partially-separated conductor 414 is shown bent away from the remaining portion of the multi-conductor ribbon 402 to isolate the partially separated conductor 414 from the remaining conductors 412. In some embodiments, individual conductors can be partially separated to facilitate the electrical coupling of a specific conductor to one or more corresponding electrodes disposed on a distal end of a lead, or to one or more corresponding terminals disposed on a proximal end of a lead. In other embodiments, individual conductors can be partially separated to distinguish one or more conductors from other conductors, for example, when troubleshooting an electrical malfunction. Additionally, individual conductors can be partially separated to facilitate wrapping or shaping the multi-conductor ribbon 402.

In at least some embodiments, the insulation may be ablated at selected locations to expose one or more underlying conductors for electrically coupling the one or more exposed conductors to terminals or electrodes when the multi-conductor ribbon is disposed in a lead. In some embodiments, conductor-exposure sites may include discrete ablated portions through the insulation at or near one or both of the ends. FIG. 7A is a schematic perspective view of one embodiment of the multi-conductor ribbon 402 with conductors 414, 504, and 702-707 exposed through ablated portions of the insulation 416.

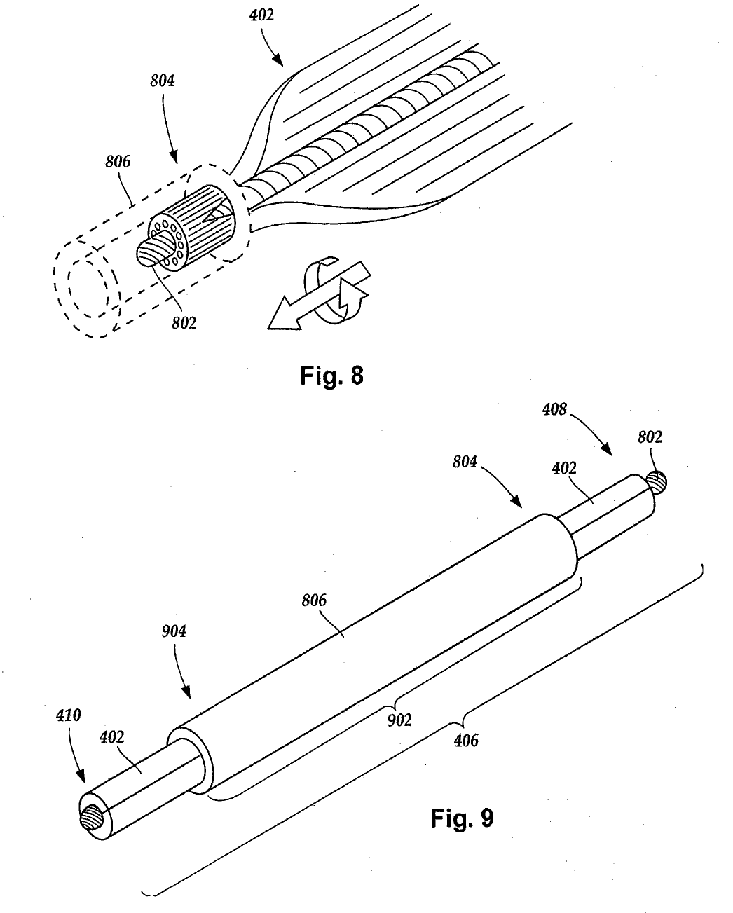

The locations of the conductor-exposure sites may vary, depending on the selected configuration of the multi-conductor ribbon 402 within a lead. In FIG. 7A, the conductor-exposure sites are configured and arranged to align with corresponding electrodes (see e.g., 134 of FIG. 2), or terminals (see e.g., 310 in FIG. 3A or 336 in FIG. 3B), when the multi-conductor ribbon 402 is inserted into a lead. In other embodiments, the entire insulation can be ablated from one or more of the ends. FIG. 7B shows the multi-conductor ribbon 402 with the insulation 416 ablated from the second end 410 to facilitate electrically coupling the conductors 412 to one or more terminals or electrodes when the multi-conductor ribbon 402 is disposed in a lead. Conductors can be exposed through the insulation by other processes including, for example, thermal processes (e.g., melting the insulation), mechanical processes (e.g., grit blasting, abrasion, or stripping the insulation), and the like or combinations thereof. In at least some embodiments, the multi-conductor ribbon 402 is inserted into a lead by the method described below, with reference to FIGS. 8 and 9.

In at least some embodiments, the multi-conductor ribbon 402 may be inserted into a lead. In at least some embodiments, the multi-conductor ribbon 402 is rolled longitudinally to form a tube that can be inserted into an outer lead covering of a lead. In at least some embodiments, a mandrel may be used to facilitate insertion of a multi-conductor ribbon into an outer lead covering. For example, in some embodiments the width of the multi-conductor ribbon is wrapped around a circumference of a mandrel and inserted into an outer lead covering. FIG. 8 is a schematic perspective view of one embodiment of the multi-conductor ribbon 402 partially wrapped around a tubular-shaped mandrel 802 and partially inserted into a proximal end 804 of a cylindrical outer lead covering 806. The multi-conductor ribbon 402 is wrapped around the mandrel 802 so that the longitudinal lengths of the conductors extend approximately along a longitudinal length of the outer lead covering 806. In alternate embodiments, the multi-conductor ribbon 402 is inserted into a distal end of the outer lead covering 806. In at least some embodiments, the mandrel may be removed after the multi-conductor ribbon 402 is disposed in the lead body 802. In at least some embodiments, the multi-conductor ribbon 402 may be inserted into the outer lead covering 806 without using the mandrel 802.

In at least some embodiments, the multi-conductor ribbon 402 is inserted into the outer lead covering 806 so that the conductors disposed in the multi-conductor ribbon 402 are approximately evenly spaced in a single layer around an inner surface of the lead. FIG. 9 is a schematic perspective view of one embodiment of the multi-conductor ribbon 402 fully inserted into the outer lead covering 806. In at least some embodiments, the longitudinal length 406 of the multi-conductor ribbon 402 is greater than the longitudinal length 902 of the outer lead covering 806. Thus, in at least some embodiments, the multi-conductor ribbon 402 can be inserted into the outer lead covering 806 so that the first end 408 of the multi-conductor ribbon 402 can extend from the proximal end 804 of the outer lead covering 806 while the second end 410 of the multi-conductor ribbon 402 extends from a distal end 904 of the outer lead covering 806.

In at least some embodiments, terminals (see e.g., 310 of FIGS. 3A and 336 of FIG. 3B) can be disposed over the exposed first end 408 of the multi-conductor ribbon 402 and terminals (see e.g., 134 of FIGS. 1 and 2) can be disposed over the exposed second end 410 of the multi-conductor ribbon 402. In at least some embodiments, conductor-exposure sites can be formed at selected locations, as shown in FIGS. 7A and 7B, to facilitate electrical coupling of selected conductors to selected terminals disposed over the multi-conductor ribbon 402 at the first end 408, and also to facilitate electrical coupling of selected conductors to selected electrodes disposed over the multi-conductor ribbon 402 at the second end 410.

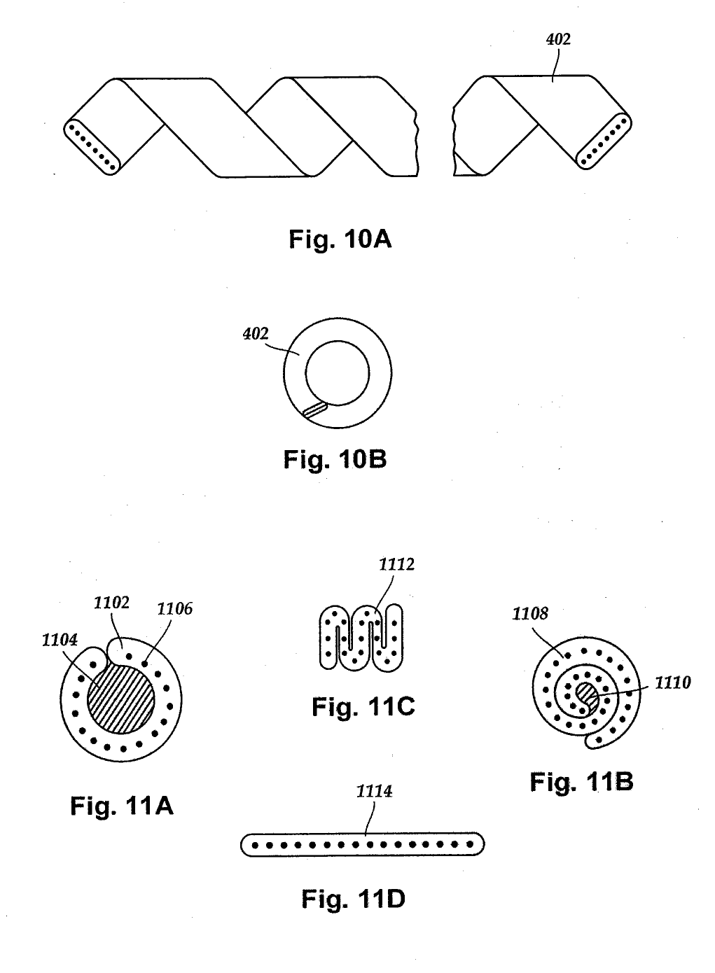

In alternate embodiments, the multi-conductor ribbon 402 is inserted into an outer lead covering in other orientations besides rolling the multi-conductor ribbon 402 into a tube. For example, in some embodiments, the multi-conductor ribbon 402 is coiled and placed in an outer lead covering. FIG. 10A is a schematic side view of one embodiment of the multi-conductor ribbon 402 placed in a coiled position. FIG. 10B is a schematic end view of the multi-conductor ribbon 410 placed in a coiled position. In at least some embodiments, a mandrel may be used to facilitate insertion of the coiled multi-conductor ribbon 402 into an outer lead covering. It may be an advantage in some situations to insert the multi-conductor ribbon 402 into an outer lead covering in a coiled position. For example, magnetic-resonance-imaging-safe leads sometimes utilize one or more coiled sections.

In at least some embodiments, more than eight conductors are disposed in a multi-conductor ribbon. As discussed above, multi-conductor ribbons may contain many different numbers of conductors. FIG. 11A is a schematic end view of one embodiment of a multi-conductor ribbon 1102 wrapped around a mandrel 1104 in a manner similar to the way the multi-conductor ribbon (402 in FIGS. 8 and 9) is wrapped around the mandrel (802 in FIGS. 8 and 9). The multi-conductor ribbon 1102 includes sixteen conductors, such as conductor 1106. In other embodiments, a multi-conductor ribbon may be rolled to form a tube with multiple layers of conductors. FIG. 11B is a schematic end view of one embodiment of a multi-conductor ribbon 1108 rolled more than one time around a mandrel 1110. Note that the embodiment of the multi-conductor ribbon 1108 shown in FIG. 11B can also be rolled without the use of a mandrel.

In at least some embodiments, a multi-conductor ribbon may be configured and arranged into many different possible shapes for insertion into a lead body. FIG. 11C is a schematic end view of one embodiment of a multi-conductor ribbon 1112 folded repeatedly in an accordion-like manner along the longitudinal length of the multi-conductor ribbon 1112. Placing a multi-conductor ribbon into a folded or rolled configuration, as shown in FIGS. 11B and 11C may offer an advantage of having a smaller cross-sectional area than the cross-sectional areas of the multi-conductor ribbons shown in FIGS. 9-11A.

FIG. 11D is a schematic end view of one embodiment of a multi-conductor ribbon 1114 in a flat configuration. Disposing the multi-conductor ribbon 1114 in a correspondingly flat lead may offer the advantage of decreased patient post-implantation discomfort due to a reduced subcutaneous profile of the lead in at least one plane, when compared to the subcutaneous profiles of similarly-sized multi-conductor ribbons configured and arranged for insertion into leads with circular cross-sectional shapes. In at least some embodiments, a multi-conductor ribbon may be shaped into a plurality of different configurations along the longitudinal length of the multi-conductor ribbon so that the multi-conductor ribbon has two or more different cross-sectional shapes along different portions of the longitudinal length of the multi-conductor ribbon.

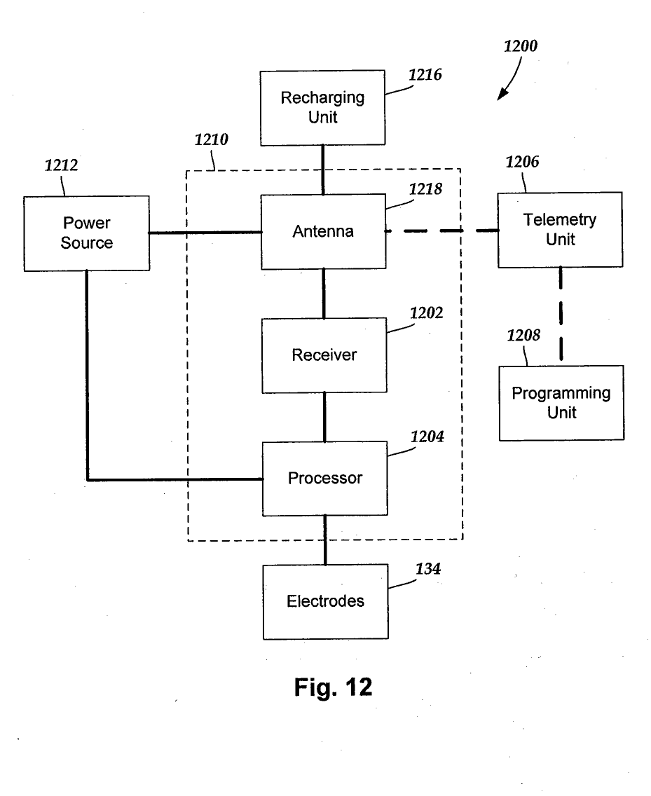

FIG. 12 is a schematic overview of one embodiment of components of an electrical stimulation system 1200 including an electronic subassembly 1210 disposed within a control module. It will be understood that the electrical stimulation system can include more, fewer, or different components and can have a variety of different configurations including those configurations disclosed in the stimulator references cited herein.

Some of the components (for example, power source 1212, antenna 1218, receiver 1202, and processor 1204) of the electrical stimulation system can be positioned on one or more circuit boards or similar carriers within a sealed housing of an implantable pulse generator, if desired. Any power source 1212 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Patent Application Publication No. 2004/0059392, incorporated herein by reference.

As another alternative, power can be supplied by an external power source through inductive coupling via the optional antenna 1218 or a secondary antenna. The external power source can be in a device that is mounted on the skin of the user or in a unit that is provided near the user on a permanent or periodic basis.

If the power source 1212 is a rechargeable battery, the battery may be recharged using the optional antenna 1218, if desired. Power can be provided to the battery for recharging by inductively coupling the battery through the antenna to a recharging unit 1216 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. A processor 1204 is generally included to control the timing and electrical characteristics of the electrical stimulation system. For example, the processor 1204 can, if desired, control one or more of the timing, frequency, strength, duration, and waveform of the pulses. In addition, the processor 1204 can select which electrodes can be used to provide stimulation, if desired. In some embodiments, the processor 1204 may select which electrode(s) are cathodes and which electrode(s) are anodes. In some embodiments, the processor 1204 may be used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 1208 that, for example, allows modification of pulse characteristics. In the illustrated embodiment, the processor 1204 is coupled to a receiver 1202 which, in turn, is coupled to the optional antenna 1218. This allows the processor 1204 to receive instructions from an external source to, for example, direct the pulse characteristics and the selection of electrodes, if desired.

In one embodiment, the antenna 1218 is capable of receiving signals (e.g., RF signals) from an external telemetry unit 1206 which is programmed by a programming unit 1208. The programming unit 1208 can be external to, or part of, the telemetry unit 1206. The telemetry unit 1206 can be a device that is worn on the skin of the user or can be carried by the user and can have a form similar to a pager, cellular phone, or remote control, if desired. As another alternative, the telemetry unit 1206 may not be worn or carried by the user but may only be available at a home station or at a clinician's office. The programming unit 1208 can be any unit that can provide information to the telemetry unit 1206 for transmission to the electrical stimulation system 1200. The programming unit 1208 can be part of the telemetry unit 1206 or can provide signals or information to the telemetry unit 1206 via a wireless or wired connection. One example of a suitable programming unit is a computer operated by the user or clinician to send signals to the telemetry unit 1206.

The signals sent to the processor 1204 via the antenna 1218 and receiver 1202 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 1200 to cease operation, to start operation, to start charging the battery, or to stop charging the battery. In other embodiments, the stimulation system does not include an antenna 1218 or receiver 1202 and the processor 1204 operates as programmed.

Optionally, the electrical stimulation system 1200 may include a transmitter (not shown) coupled to the processor 1204 and the antenna 1218 for transmitting signals back to the telemetry unit 1406 or another unit capable of receiving the signals. For example, the electrical stimulation system 1200 may transmit signals indicating whether the electrical stimulation system 1200 is operating properly or not or indicating when the battery needs to be charged or the level of charge remaining in the battery. The processor 1204 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. A lead assembly comprising:
a lead with a distal end, a proximal end, and a longitudinal length, the lead comprising
a plurality of electrodes disposed at the distal end,
a plurality of terminals disposed at the proximal end, and
an outer lead covering extending along the longitudinal length of the lead from a region proximal to the plurality of electrodes to a region distal to the plurality of terminals; and
a multi-conductor ribbon disposed within the outer lead covering, the multi-conductor ribbon having a first end, a second end, a width, and a longitudinal length, the multi-conductor ribbon comprising a plurality of conductors and a non-conductive insulation, the plurality of conductors aligned longitudinally along the multi-conductor ribbon and the non-conducting insulation encasing and insulating each of the conductors except for proximal and distal ends of the conductors, each conductor electrically coupling at least one terminal to at least one electrode.

2. The lead assembly of claim 1, wherein the non-conductive insulation further comprises at least one weakened region extending along at least a portion of the longitudinal length of the multi-conductor ribbon between two adjacent conductors.

3. The lead assembly of claim 2, wherein the at least one weakened region is disposed on the first end of the multi-conductor ribbon.

4. The lead assembly of claim 2, wherein the at least one weakened region is formed as at least one perforation, depression, striation, or groove.

5. The lead assembly of claim 1, further including at least one conductor-exposure site, the at least one conductor-exposure site comprising a removed portion of the non-conductive insulation exposing at least one underlying conductor.

6. The lead assembly of claim 5, wherein at least one conductor-exposure site aligns with at least one of the plurality of electrodes or the plurality of terminals.

7. The lead assembly of claim 5, wherein at least one conductor-exposure site is disposed on the first end.

8. The lead assembly of claim 1, wherein the multi-conductor ribbon has a substantially tubular shape.

9. The lead assembly of claim 8, wherein the substantially tubular-shaped multi-conductor ribbon comprises a single layer of conductors.

10. The lead assembly of claim 8, wherein the substantially tubular-shaped multi-conductor ribbon comprises multiple layers of conductors.

11. The lead assembly of claim 1, wherein the multi-conductor ribbon is folded in an accordion-like configuration.

12. The lead assembly of claim 1, wherein the multi-conductor ribbon is flexible.

13. An electrical stimulating system comprising:
a lead with a distal end, a proximal end, and a longitudinal length, the lead comprising
a plurality of electrodes disposed at the distal end,
a plurality of terminals disposed at the proximal end, and
an outer lead covering extending along the longitudinal length of the lead from a region proximal to the plurality of electrodes to a region distal to the plurality of terminals;
a multi-conductor ribbon disposed within the outer lead covering, the multi-conductor ribbon having a first end, a second end, a width, and a longitudinal length, the multi-conductor ribbon comprising a plurality of conductors and a non-conductive insulation, the plurality of conductors aligned longitudinally along the multi-conductor ribbon and the non-conducting insulation encasing and insulating each of the conductors except for proximal and distal ends of the conductors, each conductor electrically coupling at least one terminal to at least one electrode;
a control module configured and arranged to electrically couple to electrodes of the lead, the control module comprising
a housing, and
an electronic subassembly disposed in the housing; and
a connector for receiving the lead, the connector comprising
a connector housing defining a first port for receiving the proximal end of the lead, and
a plurality of connector contacts disposed in the connector housing, the connector contacts configured and arranged to couple to the terminals disposed at the proximal end of the lead.

14. The electrical stimulating system of claim 13, further including a lead extension having a proximal end and a distal end, the connector disposed on the distal end of the lead extension.

15. The electrical stimulating system of claim 14, wherein the proximal end of the lead extension is configured and arranged for insertion into another connector.

16. The electrical stimulating system of claim 13, wherein the connector is disposed on the control module.

17. A method for forming a lead, the method comprising:
disposing a multi-conductor ribbon with a first end and a second end into an outer lead covering, the multi-conductor ribbon comprising a plurality of conductors extending along the multi-conductor ribbon and separated from one another by insulation;
removing portions of the insulation to expose each of the conductors at both the first end and at the second end of the multi-conductor ribbon;
electrically coupling at least one terminal to each of the conductors exposed at the first end of the multi-conductor ribbon; and
electrically coupling at least one electrode to each of the conductors exposed at the second end of the multi-conductor ribbon.

18. The method of claim 17, wherein disposing a multi-conductor ribbon with a first end and a second end into an outer lead covering comprises rolling the multi-conductor ribbon into a tube.

19. The method of claim 17, wherein disposing a multi-conductor ribbon with a first end and a second end into an outer lead covering comprises folding the multi-conductor ribbon in an accordion-like manner.

20. The method of claim 17, wherein disposing a multi-conductor ribbon with a first end and a second end into an outer lead covering comprises using a mandrel to facilitate insertion of the multi-conductor ribbon into the outer lead covering.
